SEQUENCE LISTING (4 sequences)

SEQ ID NO: 1 - RNA, 36 nt, synthetic construct
  source 1..36: mol_type = other RNA
tcaaaatggaaggttatacagcagccgaaaggctgc

SEQ ID NO: 2 - RNA, 22 nt, synthetic construct
  source 1..22: mol_type = other RNA
tgtataaccttccattttgagg

SEQ ID NO: 3 - RNA, 36 nt, synthetic construct; identical to SEQ ID NO: 1
  modified_base 1: mod_base = OTHER; note = 2'-O-methyluridine with a 3'-phosphorothioate linkage
  modified_base 2: mod_base = cm
  modified_base 3: mod_base = OTHER; note = 2'-O-methyladenosine
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methyladenosine
  modified_base 7: mod_base = um
  modified_base 8: mod_base = OTHER; note = 2'-fluoroguanosine
  modified_base 9: mod_base = OTHER; note = 2'-fluoroguanosine
  modified_base 10: mod_base = OTHER; note = 2'-fluoroadenosine
  modified_base 11: mod_base = OTHER; note = 2'-fluoroadenosine
  modified_base 12: mod_base = gm
  modified_base 13: mod_base = gm
  modified_base 14: mod_base = um
  modified_base 15: mod_base = um
  modified_base 16: mod_base = OTHER; note = 2'-O-methyladenosine
  modified_base 17: mod_base = um
  modified_base 18: mod_base = OTHER; note = 2'-O-methyladenosine
  modified_base 19: mod_base = cm
  modified_base 20: mod_base = OTHER; note = 2'-O-methyladenosine
  modified_base 21: mod_base = gm
  modified_base 22: mod_base = cm
  modified_base 23: mod_base = OTHER; note = 2'-O-methyladenosine
  modified_base 24: mod_base = gm
  modified_base 25: mod_base = cm
  modified_base 26: mod_base = cm
  modified_base 27: mod_base = gm
  modified_base 28: mod_base = OTHER; note = 2'-aminodiethoxymethanol-Adenine-GalNAc
  modified_base 29: mod_base = OTHER; note = 2'-aminodiethoxymethanol-Adenine-GalNAc
  modified_base 30: mod_base = OTHER; note = 2'-aminodiethoxymethanol-Adenine-GalNAc
  modified_base 31: mod_base = gm
  modified_base 32: mod_base = gm
  modified_base 33: mod_base = cm
  modified_base 34: mod_base = um
  modified_base 35: mod_base = gm
  modified_base 36: mod_base = cm
  source 1..36: mol_type = other RNA

SEQ ID NO: 4 - RNA, 22 nt, synthetic construct
  modified_base 1: mod_base = OTHER; note = MePhosphonate-4O-2'-O-methyl modified uridine with a 3'-phosphorothioate linkage
  modified_base 2: mod_base = OTHER; note = 2'-fluoroguanosine with a 3'-phosphorothioate linkage
  modified_base 3: mod_base = OTHER; note = 2'-fluorouridine with a 3'-phosphorothioate linkage
  modified_base 4: mod_base = OTHER; note = 2'-fluoroadenosine
  modified_base 5: mod_base = OTHER; note = 2'-fluorouridine
  modified_base 6: mod_base = OTHER; note = 2'-O-methyladenosine
  modified_base 7: mod_base = OTHER; note = 2'-fluoroadenosine
  modified_base 8: mod_base = cm
  modified_base 9: mod_base = cm
  modified_base 10: mod_base = OTHER; note = 2'-fluorouridine
  modified_base 11: mod_base = um
  modified_base 12: mod_base = cm
  modified_base 13: mod_base = cm
  modified_base 14: mod_base = OTHER; note = 2'-fluoroadenosine
  modified_base 15: mod_base = um
  modified_base 16: mod_base = um
  modified_base 17: mod_base = um
  modified_base 18: mod_base = um
  modified_base 19: mod_base = gm
  modified_base 20: mod_base = OTHER; note = 2'-O-methyladenosine with a 3'-phosphorothioate linkage
  modified_base 21: mod_base = OTHER; note = 2'-O-methylguanosine with a 3'-phosphorothioate linkage
  modified_base 22: mod_base = gm
  source 1..22: mol_type = other RNA
tgtataaccttccattttgagg

(19) United States
(12) Patent Application Publication
(10) Pub. No.: US 2026/0286335 A1
(43) Pub. Date: Sep. 24, 2026

(54) APPARATUSES FOR AND METHODS OF CONCENTRATING BIOMOLECULES

(71) Applicant: Eli Lilly and Company, Indianapolis, IN (US)

(72) Inventors: McKensie Lee MASON, Greenwood, IN (US); Ken Kangyi QIAN, Carmel, IN (US); Yufei WEI, Indianapolis, IN (US)

(21) Appl. No.: 19/476,745

(22) PCT Filed: Apr. 25, 2024
(86) PCT No.: PCT/US2024/026206

§ 371 Date: Oct. 20, 2025

Related U.S. Application Data
(60) Provisional application No. 63/498,279, filed on Apr. 26, 2023.

(51) Int. Cl. C12N 15/10

(52) U.S. Cl. CPC C12N 15/1003

(57) ABSTRACT

Methods are described for concentrating a biomolecule-containing solution, such as an oligonucleotide-containing solution, to a concentration ≥about 200 mg/mL from an initial concentration of about ≤20 mg/mL, via thin film evaporation (TFE), for high dose/low volume applications. Apparatuses also are described for concentrating a biomolecule-containing solution as described herein.

### REFERENCE TO SEQUENCE LISTING SUBMITTED ELECTRONICALLY

The disclosure is being filed along with a Sequence Listing in ST.26 XML format. The Sequence Listing is provided as a file titled “30289_US_PRI” created 22 Feb. 2024 and is 29.9 kilobytes (kb) in size. The Sequence Listing information in the ST.26 XML format is incorporated herein by reference in its entirety.

### TECHNICAL FIELD

The disclosure relates to chemistry and engineering, and more particularly it relates to apparatuses for and methods of concentrating biomolecules in solutions, such as single-stranded (ss) or double-stranded (ds) oligonucleotides (i.e., therapeutic oligonucleotides), for use as a drug substance (DS) in the manufacture of a drug product (DP).

### BACKGROUND

Therapeutic oligonucleotides are a newer modality for treating and preventing diseases and disorders. Therapeutic oligonucleotide synthesis may involve a number of upstream and downstream steps such as, for example, synthesizing, cleaving and deprotecting, purifying, and concentrating. Of particular interest herein is the concentrating of therapeutic oligonucleotides in solution for use as a DS in the manufacture of a DP or other pharmaceutical composition.

A number of methods of concentrating oligonucleotide-containing solutions are known such as chromatography, dialysis, evaporation, precipitation and ultrafiltration/diafiltration (UF/DF). At present, these methods of concentrating oligonucleotide-containing solutions do not reliably achieve final oligonucleotide concentrations in solution greater than about 100 mg/mL. Highly concentrated oligonucleotide-containing solutions, however, are required for formulating and dosing individuals in need thereof with a minimal volume of solution.

Therefore, there is a need for apparatuses for and methods of concentrating biomolecule-containing solutions, such as oligonucleotide-containing solutions, to a concentration ≥200 mg/mL.

### BRIEF SUMMARY

To address this need, the disclosure first describes a method of concentrating an oligonucleotide-containing solution that includes at least steps of:
(a). flowing the solution through an evaporation vessel, where the solution has an initial oligonucleotide concentration of ≤about 20 mg/mL, where the evaporation vessel includes a rotatable blade, a means for heating and a means for pressurizing, where the rotatable blade wipes the solution into a thin film around an internal surface of the evaporation vessel, and where the evaporation vessel can be at an initial temperature and at an initial pressure and;
(b). passing a gas through the evaporation vessel;
(c). condensing and collecting a volatile liquid from the solution in a condenser in fluid communication with the evaporation vessel, where the condenser can be at an initial temperature; and
(d). repeating steps (a) to (c) until the solution can have a final oligonucleotide concentration of ≥about 200 mg/mL.

In some instances, the oligonucleotide-containing solution can be at a flow rate from about 0.5 mL/min to about 4.5 mL/min.

In some instances, the rotating blade can rotate at a speed of about 15 rpm to about 105 rpm.

In some instances, the initial temperature of the evaporation vessel can be about 35° C. to about 60° C.

In some instances, the initial pressure of the evaporation vessel can be about 10 torr to about 65 torr.

In some instances, the gas can be an inert gas such as nitrogen and can be at an initial sweep pressure of about 1.5 psi to about 5 psi.

In some instances, the condenser can be an internal condenser (i.e., within the evaporation vessel). In other instances, the condenser can be an external condenser (i.e., separate from the evaporation vessel).

In some instances, the initial temperature of the condenser can be about 0° C.

Second, the disclosure describes a method of concentrating an oligonucleotide-containing solution that includes at least steps of:
(a). loading an initial volume of the solution into an evaporation vessel, where the solution can have an initial oligonucleotide concentration of ≤about 20 mg/mL, and where the evaporation vessel can be at an initial pressure and at an initial temperature;
(b). boiling the solution in the evaporation vessel for a period of time; and
(c). drawing water vapor from the evaporation vessel via a condenser, where the condenser can be at an initial temperature, whereby the solution can have a final oligonucleotide concentration of ≥about 200 mg/mL.

In some instances, the initial volume of oligonucleotide-containing solution is about 1 mL to about 10 mL.

In some instances, the initial pressure of the evaporation vessel can be about 20 torr to about 30 torr.

In some instances, the initial temperature of the evaporation vessel can be about 55° C. to about 65° C.

In some instances, the period of time can be about 4 min to about 4.5 min.

In some instances, the initial temperature of the condenser can be about 0° C.

In some instances, the method further can include a step of:
(d). transferring the solution having a final oligonucleotide concentration of ≥about 200 mg/mL from the evaporation vessel to, for example, a collection vessel.

In some instances, the method further can include a step of:
(e) repeating steps (a) to (d) on a fresh volume of the oligonucleotide-containing solution.

In some instances in the methods above, the oligonucleotide-containing solution can be a ss oligonucleotide-containing solution. In other instances in the methods above, the oligonucleotide-containing solution can be a ds oligonucleotide-containing solution.

In some instances in the methods above, the final oligonucleotide concentration can be about 200 mg/mL to about 400 mg/mL. In other instances in the methods above, the final oligonucleotide concentration can be ≤about 450 mg/mL. In yet other instances in the methods above, the final oligonucleotide concentration can be ≤about 500 mg/mL.

In some instances in the methods above, the oligonucleotide-containing solution can be at a final volume of ≤about 2 mL.

Moreover, the methods above optionally may include a subsequent step of performing tangential flow filtration (TFF) or lyophilization on the solution for additional concentrating and/or purifying.

Third, the disclosure describes a composition including oligonucleotides, such as ds oligonucleotides, at a concentration of ≥about 200 mg/mL.

Alternatively, the disclosure describes a composition resulting from the methods above having a concentration of ≥about 200 mg/mL.

In some instances of these compositions, the composition is a solution. In other instances, the composition is a lyophilized powder.

In some instances of these compositions, especially when a solution, the solution can be at a final volume of ≤about 2 mL.

Fourth, the disclosure first describes an apparatus for concentrating a biomolecule-containing solution by intermittent evaporation (IE), such as an oligonucleotide-containing solution, for high dose/low volume administration. The apparatus includes:
(i). an evaporation vessel, where the evaporation vessel includes a means for heating and a first means for pressurizing;
(ii). a first reservoir in fluid communication with the evaporation vessel, where the first reservoir is a feed for an unconcentrated biomolecule-containing solution;
(iii). a second reservoir in fluid communication with the evaporation vessel, where the second reservoir is a collector for a concentrated biomolecule-containing solution; and (iv). a condenser in fluid communication with the evaporation vessel, where the condenser includes a means for cooling and a second means for pressurizing.

An advantage of the methods herein is that they can be used to concentrate ss or ds oligonucleotides.

An advantage of the methods herein is that they can be used to significantly increase oligonucleotide concentration in a solution as compared to a concentration that can be achieved in conventional wiped film evaporation (WFE) or TFF setups.

An advantage of the methods herein is that they provide no measurable loss of chemical purity of oligonucleotides in oligonucleotide-containing solutions (i.e., do not degrade the oligonucleotide).

An advantage of the methods herein is that they result in a reduce volume for efficient storage and potential high dosing requirements.

An advantage of the methods herein is that they can be combined with other methods of concentrating oligonucleotides (e.g., TFF or lyophilization) to achieve higher concentrations thereof.

### BRIEF DESCRIPTION OF THE DRAWINGS

The advantages, effects, features and objects other than those set forth above will become more readily apparent when consideration is given to the detailed description below. Such detailed description refers to the following drawing(s), where:

FIG. 1 shows an exemplary apparatus for concentrating an oligonucleotide-containing solution via WFE.

FIG. 2 shows an exemplary apparatus for concentrating an oligonucleotide-containing solution via IE.

FIGS. 3A-B show overlaid denaturing ultra-performance liquid chromatography (UPLC) chromatograms of oligonucleotide-containing solutions, where FIG. 3A shows the results of concentrating via TFF only, concentrating via TFF followed by WFE, concentrating via WFE only and concentrating via WFE followed by WFE (“double-pass WFE”), and where FIG. 3B shows the results of concentrating via IE.

FIGS. 4A-B show overlaid non-denaturing UPLC chromatograms of oligonucleotide-containing solutions, where FIG. 4A shows the results of concentrating via TFF only, concentrating via TFF followed by WFE, concentrating via WFE only and concentrating via “double-pass” WFE, and where FIG. 4B shows the results of concentrating via IE.

### DETAILED DESCRIPTION

### Overview

Therapeutic oligonucleotides, especially activating RNA (aRNA)-, editing RNA (eRNA)-, inhibiting RNA (iRNA)- and messenger RNA (mRNA)-based therapeutic oligonucleotides, are an emerging class of biomolecules. While lyophilization typically is a final step in preparing DS to produce a solid powder, use of a DS solution of therapeutic oligonucleotides may be beneficial due to their high stability in liquid or frozen solutions and to streamline the manufacturing process while making a DP solution. For example, an end step of DS preparation can be to remove any aqueous solution via lyophilization to form a solid powder, and a DP process in turn can include dissolving the DS (as a solid powder/lyophilizate) into water to form the DP. As such, alternatives to lyophilization are needed that allow for a more seamless transition from DS to DP and that eliminate the costs and time required for lyophilization.

With conventional TFF, ds oligonucleotide-containing solution concentrations >100 mg/mL are difficult to achieve due to a decline in permeate flux, which can be due to increases in osmotic pressure, viscosity and/or fouling at the membrane surface. While conventional WFE does not have the same membrane-based challenges as TFF, there can be issues with gelation or drying of the oligonucleotide in the evaporation vessel (i.e., a column) that can occur when conditions favor slow feed flow rates or with a large degree of evaporation to target very high concentration factors during one pass through the vessel.

In contrast, and as shown in the Examples below, a “double-pass” WFE method herein and an IE method herein achieved concentrations >about 200 mg/mL. With regard to the IE method, avoiding the “wiped film” element of WFE may help to alleviate or suppress gel and solid formation from over-evaporation of the thin film, thereby allowing for concentrating in a single pass through the system, as well as avoiding a possibility of wiper blade degradation. The apparatuses and methods herein can provide high concentration therapeutic oligonucleotides (i.e., >about 200 mg/mL) in a low volume solution (i.e., ≤about 2 mL).

### ABBREVIATIONS AND DEFINITIONS

Unless defined otherwise, all technical and scientific terms used herein have the same meaning as commonly understood by one of skill in the art to which the disclosure pertains. Although any methods and materials similar to or equivalent to those described herein can be used in the practice or testing of the methods herein, the preferred methods and materials are described herein.

Additionally, reference to an element by the indefinite article “a” or “an” does not exclude the possibility that more than one element is present, unless the context clearly requires that there be one and only one element. The indefinite article “a” or “an” thus usually means “at least one.”

Moreover, use of “including,” as well as other forms, such as “including but not limited, “include,” “includes” and “included,” is not limiting.

Certain abbreviations used herein are as follows:

“ADAR” refers to adenosine deaminase acting on RNA enzyme; “API” refers to active pharmaceutical ingredient; “aRNA” refers to activating RNA; “ASO” refers to antisense oligonucleotide; “DF” refers to dilution factor; “DIPEA” refers to N,N-diisopropylethylamine: “DIW” refers to deionized water; “DNA” refers to deoxyribonucleic acid; “DP” refers to drug product; “ds” refers to double-stranded”; “DS” refers to drug substance; “DsiRNA” refers to Dicer substrate interfering RNA; “eRNA” refers to editing RNA; “GalNAc” refers to N-acetylgalactosamine; “H₂O” refers to water; “HFIP” refers to hexafluoroisopropanol; “hr” refers to hour(s); “iRNA” refers to inhibiting RNA; “IE” refers to intermittent evaporation; “kDa” refers to kilodalton(s); “L” refers to liter(s); “MEC” refers to molar extinction coefficient; “mg” refers to milligram; “min” refers to minute(s); “mL” refers to milliliter(s); “mol” refers to moles; “mRNA” refers to messenger RNA; “miRNA” refers to microRNA; “MW” refers to molecular weight; “MWCO” refers to molecular weight cutoff; “PES” refers to polyethersulfone; “PFA” refers to perfluoroalkoxy; “psi” refers to pounds per square inch; “RISC” refers to RNA-induced silencing complex; “RITA” refers to RNA-induced transcriptional activation; “RNA” refers to ribonucleic acid; “rRNA” refers to ribosomal RNA; “rpm” refers to revolutions per minute; “shRNA” refers to short hairpin RNA; “siRNA” refers to small interfering RNA; “SPS” refers to solid-phase synthesis; “ss” refers to single-stranded; “TFE” refers to thin film evaporation; “TFF” refers to tangential flow filtration; “TMP” refers to transmembrane pressure; “tRNA” refers to transfer RNA; “UPLC” refers to ultra-performance liquid chromatography; “UV” refers to ultraviolet; “V” refers to volume; “W” refers to weight; and “WFE” refers to wiped film evaporation.

Certain definitions used herein are defined as follows:

As used herein, “about” means within a statistically meaningful range of a value or values such as, for example, a stated concentration, flow rate, length, molecular weight, pH, pressure, sequence similarity, speed, time frame, temperature, volume, etc. Such a value or range can be within an order of magnitude typically within 20%, more typically within 10%, and even more typically within 5% of a given value or range. The allowable variation encompassed by “about” will depend upon the particular system under study, and can be readily appreciated by one of skill in the art.

As used herein, “activating RNA” or “aRNA” means a nucleic acid that contains RNA and that mediates the targeted activation of a promoter or other non-coding transcript of a RNA transcript via a RNA-induced transcriptional activation (RITA) complex pathway. aRNAs typically are ds. aRNA activates, increases, modulates or upregulates expression of a target nucleotide sequence in a cell.

As used herein, “biomolecule” and the like means a molecule or compound that includes or incorporates amino acids, carbohydrates, lipids and/or nucleotides. Examples of biomolecules of interest herein include, but are not limited to, nucleic acids (e.g., oligonucleotides and polynucleotides), peptides, polypeptides and proteins.

As used herein, “deoxyribonucleotide” means a nucleotide having a hydrogen in place of a hydroxyl at the 2′ position of its pentose sugar when compared with a ribonucleotide. A modified deoxyribonucleotide has one or more modifications or substitutions of atoms other than hydroxyl at the 2′ position, including modifications or substitutions in or of the nucleobase, sugar or phosphate group.

As used herein, “drug product” or “DP” means a finished product of any therapeutic agent, such as a therapeutic oligonucleotide, that is available in the market and that is ready to use generally, but not necessarily, in association with one or more other pharmaceutically acceptable ingredients.

As used herein, “drug substance” or “DS” means an active ingredient, such as a therapeutic oligonucleotide, that is intended to furnish pharmacological activity or other direct effect in diagnosing, curing, mitigating, treating and/or preventing disease or affecting the structure or any function of the body, but does not include intermediates used in the synthesis of such ingredient. DS also is known as an active pharmaceutical ingredient (API). A DS is used in preparing a DP.

As used herein, “editing RNA” or “eRNA” means a nucleic acid that contains RNA and that mediates inserting, deleting and even base substituting of nucleotides within a target nucleotide sequence. RNA editing has been observed in a number of different types of RNA such as, for example, mRNA, microRNA (miRNA), transfer RNA (tRNA) and ribosomal RNA (rRNA). RNA editing is enzymatically mediated either by exogenously supplying adenosine deaminase acting on RNA enzyme (ADAR) or by directing an endogenous ADAR to a specific site in a target RNA nucleotide sequence, and typically involves editing at a single nucleotide site by directing the ADAR to that site through complimentary oligonucleotides. eRNAs typically are ss.

As used herein, “inhibiting RNA” or “iRNA” means a nucleic acid that contains RNA and that mediates the targeted cleavage of a RNA transcript via RNA interference, for example, through a RNA-induced silencing complex (RISC) pathway. Some iRNAs are ss and other iRNAs are ds and have a sense strand and an antisense strand, where the sense strand and the antisense strand form a duplex. iRNA directs sequence-specific degradation of mRNA via RNA interference. iRNA attenuates, inhibits, modulates or reduces expression of a target nucleotide sequence in a cell. Examples of iRNA include, but are not limited to, an antisense oligonucleotide (ASO), Dicer substrate interfering RNA (DsiRNA), miRNA, short hairpin RNA (shRNA) or small interfering RNA (siRNA).

As used herein, “intermittent evaporation” or “IE” means a process of concentrating where a defined amount of the solution to be concentrated is loaded into a vessel that is heated and is under vacuum, and where a concentrated solution is removed before loading another batch. The concentrated material is collected in an “intermittent” process here, but it is possible that the process can be continuous.

As used herein, “nucleotide” means an organic compound having a nucleoside (a nucleobase such as, for example, adenine, cytosine, guanine, thymine or uracil, and a pentose sugar such as, for example, ribose or 2′-deoxyribose) and a phosphate group. A nucleotide can serve as a monomeric unit of nucleic acid polymers such as deoxyribonucleic acid (DNA) and ribonucleic acid (RNA).

As used herein, “oligonucleotide” means a short nucleic acid compound (e.g., a polymer of less than about 100 nucleotides in length) that may include deoxyribonucleotides (or modified deoxyribonucleotides), ribonucleotides (or modified ribonucleotides) or both. Likewise, an oligonucleotide may be ss or ds and thus may or may not have duplex regions.

As used herein, “synthetic” refers to a nucleic acid or other compound that is artificially synthesized (e.g., using a machine such as, for example, a solid phase nucleic acid synthesizer) or that is otherwise not derived from a natural source (e.g., a cell or organism) that normally produces the nucleic acid or other compound.

As used herein, “ribonucleotide” means a nucleotide having a ribose as its pentose sugar, which contains a hydroxyl group at its 2′ position. A modified ribonucleotide is a ribonucleotide having one or more modifications or substitutions of atoms other than hydrogen at the 2′ position, including modifications or substitutions in or of the nucleobase, sugar or phosphate group.

As used herein, “therapeutic oligonucleotide” means a ss or ds nucleic acid that has a therapeutic application (i.e., application in treating a disease). Such a nucleic acid typically contains one or more modified nucleotide residues or linkages and also can include a targeting ligand and/or delivery vehicle. Examples of therapeutic oligonucleotides include, but are not limited to, aRNA, eRNA, iRNA and mRNA. Specific examples of therapeutic oligonucleotides include, but are not limited to, ASOs, aptamers, short activating RNAs (saRNAs), siRNAs, miRNAs and decoys.

As used herein, “wiped film evaporation” or “WFE” means a process of concentrating that utilizes a controlled flow of a low-concentration feed solution (i.e., an oligonucleotide-containing solution) down a column that is heated and under reduced pressure, while a rotating blade wipes the solution into a thin film around the internal surface of the column, which facilitates evaporation of a volatile liquid (e.g., H₂O) from the solution. Likewise, and as used herein, “double-pass WFE” means a process in which WFE is carried out at least twice on the same ds oligonucleotide-containing solution.

### Apparatuses

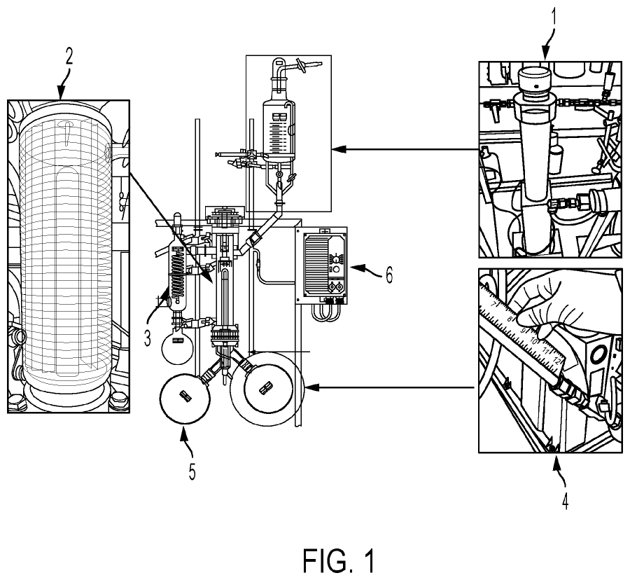

An exemplary WFE setup of this disclosure is shown in FIG. 1. Here, a low-concentration ss or ds oligonucleotide-containing solution to be concentrated (not shown) can be placed/stored in a first reservoir (1) that is in fluid communication with an evaporation vessel (2) for WFE as is known in the art (i.e., including, for example, a heating means and a rotatable wiper). The evaporation vessel (2) further can be in fluid communication with a condenser (3) for removing volatile liquid, a gas sweep source (not shown) for alleviating condensation forming within the apparatus, and a second reservoir (4) for collecting concentrated oligonucleotide-containing solution. A vacuum can be applied to the apparatus via a vacuum pump and controller (6). In some instances, the condenser can be an external condenser (as shown in FIG. 1), although an internal condenser also can be used. An optional collection vessel (5) can be used for evaporated water or solvent if an internal condenser is used.

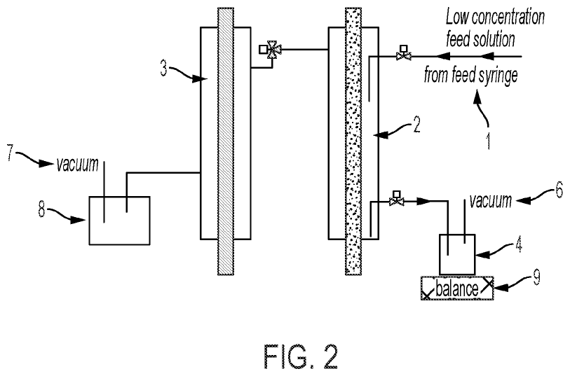

An exemplary IE setup of this disclosure is shown in FIG. 2. Here, a ss or ds oligonucleotide-containing solution to be concentrated (not shown) can be placed/stored in a first reservoir (1) (e.g., a syringe pump) in fluid communication with an evaporation vessel (2) (e.g., a column including a means for heating the column to an initial temperature and a means for pressurizing the column to an initial pressure). The evaporation vessel (2) further can be in fluid communication with a condenser (3) for removing volatile liquid and a second reservoir (4) for collecting concentrated oligonucleotide-containing solution. A vacuum can be applied to the second reservoir (4) via a vacuum pump and controller (6). The condenser (3) can be in fluid communication with a third reservoir (8) for collecting concentrated oligonucleotide-containing solution. A vacuum can be applied to the third reservoir (8) via a vacuum pump and controller (7). In some instances, the vacuum pump and controller (6) can be the same type as the vacuum pump and controller (7); however, and in other instances, the vacuum pump and controller (6) can be a distinct type from the vacuum pump and controller (7). The apparatus optionally can include a balance (9) for measuring an amount of concentrated oligonucleotide-containing solution that has been collected.

Exemplary means for heating include, but are not limited to, circulating a heated fluid, such as a gas or a liquid. In some instances, the means for heating can be a glycol/water solution.

Exemplary means for pressurizing include, but are not limited to, a vacuum.

### Methods

The methods can include the steps described herein, and these maybe be, but not necessarily, carried out in the sequence as described. Other sequences, however, also are conceivable. Moreover, individual or multiple steps may be carried out either in parallel and/or overlapping in time and/or individually or in multiply repeated steps. Furthermore, the methods may include additional, unspecified steps.

Likewise, the oligonucleotides can be prepared by any method known in the art such as, for example, solid-phase synthesis (SPS) of the individual strands, which then optionally can undergo additional steps for purifying, solvent exchanging, de-salting and concentrating prior to and/or following annealing into a duplex in water for ds oligonucleotides.

### “Double-Pass” WFE

Briefly, a method of concentrating an oligonucleotide-containing solution via “double-pass” WFE can include a step (a) of flowing the solution through an evaporation vessel having a rotatable blade, where the evaporation vessel can be at an initial pressure and at an initial temperature, and where the rotatable blade wipes the solution into a thin film around an internal surface of the evaporation vessel.

In some instances, the oligonucleotide can be a ss oligonucleotide. In other instances, the oligonucleotide can be a ds oligonucleotide.

In some instances, the initial oligonucleotide concentration in the oligonucleotide-containing solution can be ≤about 20 mg/mL. In other instances, the initial oligonucleotide concentration can be between about 5 mg/mL to about 20 mg/mL or about 10 mg/mL to about 15 mg/mL. In yet other instances, the initial oligonucleotide concentration can be about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/L, about 18 mg/mL, about 19 mg/mL or about 20 mg/mL. In yet other instances, the initial oligonucleotide concentration can be ≥about 20 mg/mL but ≤about 30 mg/mL. In some instances, the initial oligonucleotide concentration can be ≥about 30 mg/ml as long as it is a large enough volume and low enough viscosity to flow through the system (e.g., even ≥about 100 mg/mL in appropriate instances).

In some instances, the pH of the oligonucleotide-containing solution can be about 6.0 to about 7.0. In other instances, the pH of the oligonucleotide-containing solution can be about 6.0, about 6.1, about 6.2, about 6.3, about 6.4, about 6.5, about 6.6, about 6.7, about 6.8, about 6.9 or about 7.0.

In some instances, the oligonucleotide-containing solution can be flowing through the evaporation vessel at an initial flow rate of about 0.5 mL/min to about 4.5 mL/min. In other instances, the initial flow rate can be about 0.6 mL/min to about 4.4 mL/min, about 0.7 mL/min to about 4.3 mL/min, about 0.8 mL/min to about 4.2 mL/min, about 0.9 mL/min to about 4.1 mL/min, about 1.0 mL/min to about 4.0 mL/min, about 1.1 mL/min to about 3.9 mL/min, about 1.2 mL/min to about 3.8 mL/min, about 1.3 mL/min to about 3.7 mL/min, about 1.4 mL/min to about 3.6 mL/min, about 1.5 mL/min to about 3.5 mL/min, about 1.6 mL/min to about 3.4 mL/min, about 1.7 mL/min to about 3.3 mL/min, about 1.8 mL/min to about 3.2 mL/min, about 1.9 mL/min to about 3.1 mL/min, about 2.0 mL/min to about 3.0 mL/min, about 2.1 mL/min to about 2.9 mL/min, about 2.2 mL/min to about 2.8 mL/min, about 2.3 mL/min to about 2.7 mL/min, about 2.4 mL/min to about 2.6 mL/min, or about 2.5 mL/min. In yet other instances, the initial flow rate can be about 0.5 mL/min to about 1.0 mL/min, about 1.0 mL/min to about 1.5 mL/min, about 1.5 mL/min to about 2.0 mL/min, about 2.0 mL/min to about 2.5 mL/min, about 2.5 mL/min to about 3.0 mL/min, about 3.0 mL/min to about 3.5 mL/min, about 3.5 mL/min to about 4.0 mL/min, or about 4.0 mL/min to about 4.5 mL/min. In yet other instances, the initial flow rate can be about 0.5 mL/min, about 0.6 mL/min, about 0.7 mL/min, about 0.8 mL/min, about 0.9 mL/min, about 1.0 mL/min, about 1.1 mL/min, about 1.2 mL/min, about 1.3 mL/min, about 1.4 mL/min, about 1.5 mL/min, about 1.6 mL/min, about 1.7 mL/min, about 1.8 mL/min, about 1.9 mL/min, about 2.0 mL/min, about 2.1 mL/min, about 2.2 mL/min, about 2.3 mL/min, about 2.4 mL/min, about 2.5 mL/min, about 2.6 mL/min, about 2.7 mL/min, about 2.8 mL/min, about 2.9 mL/min, about 3.0 mL/min, about 3.1 mL/min, about 3.2 mL/min, about 3.3 mL/min, about 3.4 mL/min, about 3.5 mL/min, about 3.6 mL/min, about 3.7 mL/min, about 3.8 mL/min, about 3.9 mL/min, about 4.0 mL/min, about 4.1 mL/min, about 4.2 mL/min, about 4.3 mL/min, about 4.4 mL/min or about 4.5 mL/min.

In some instances, the flow rate can be fixed (i.e., can be maintained) during the concentrating. In other instances, the flow rate can be varied during the concentrating (i.e., can be increased or can be decreased from the initial flow rate).

In some instances, the initial pressure of the evaporation vessel can be about 10 torr to about 65 torr. In other instances, the initial pressure can be about 11 torr to about 64 torr, about 12 torr to about 63 torr, about 13 torr to about 62 torr, about 14 torr to about 61 torr, about 15 torr to about 60 torr, about 16 torr to about 59 torr, about 17 torr to about 58 torr, about 18 torr to about 57 torr, about 19 torr to about 56 torr, about 20 torr to about 55 torr, about 21 torr to about 54 torr, about 22 torr to about 53 torr, about 23 torr to about 52 torr, about 24 torr to about 51 torr, about 25 torr to about 50 torr, about 26 torr to about 49 torr, about 27 torr to about 48 torr, about 28 torr to about 47 torr, about 29 torr to about 46 torr, about 30 torr to about 45 torr, about 31 torr to about 44 torr, about 32 torr to about 43 torr, about 33 torr to about 42 torr, about 34 torr to about 41 torr, about 35 torr to about 40 torr, about 36 torr to about 39 torr, or about 37 torr to about 38 torr. In yet other instances, the initial pressure can be about 10 torr to about 15 torr, about 15 torr to about 20 torr, about 20 torr to about 25 torr, about 25 torr to about 30 torr, about 30 torr to about 35 torr, about 35 torr to about 40 torr, about 40 torr to about 45 torr, about 45 torr to about 50 torr, about 50 torr to about 55 torr, about 55 torr to about 60 torr, or about 60 torr to about 65 torr. In yet other instances, the initial pressure can be about 10 torr, about 11 torr, about 12 torr, about 13 torr, about 14 torr, about 15 torr, about 16 torr, about 17 torr, about 18 torr, about 19 torr, about 20 torr, about 21 torr, about 22 torr, about 23 torr, about 24 torr, about 25 torr, about 26 torr, about 27 torr, about 28 torr, about 29 torr, about 30 torr, about 31 torr, about 32 torr, about 33 torr, about 34 torr, about 35 torr, about 36 torr, about 37 torr, about 38 torr, about 39 torr, about 40 torr, about 41 torr, about 42 torr, about 43 torr, about 44 torr, about 45 torr, about 46 torr, about 47 torr, about 48 torr, about 49 torr, about 50 torr, about 51 torr, about 52 torr, about 53 torr, about 54 torr, about 55 torr, about 56 torr, about 57 torr, about 58 torr, about 59 torr, about 60 torr, about 61 torr, about 62 torr, about 63 torr, about 64 torr or about 65 torr.

In some instances, the pressure of the evaporation vessel can be fixed (i.e., can be maintained) during the concentrating. In other instances, the pressure of the evaporation vessel can be varied during the concentrating (i.e., can be increased or can be decreased from the initial pressure).

In some instances, the initial temperature of the evaporation vessel can be about 35° C. to about 60° C. In other instances, the initial temperature can be about 36° C. to about 59° C., about 37° C. to about 58° C., about 38° C. to about 57° C., about 39° C. to about 56° C., about 40° C. to about 55° C., about 41° C. to about 54° C., about 42° C. to about 53° C., about 43° C. to about 52° C., about 44° C. to about 51° C., about 45° C. to about 50° C., about 46° C. to about 49° C., or about 47° C. to about 48° C. In yet other instances, the initial temperature can be at about 35° C. to about 40° C., about 40° C. to about 45° C., about 45° C. to about 50° C., about 50° C. to about 55° C., or about 55° C. to about 60° C. In yet other instances, the initial temperature can be about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C. or about 60° C.

In some instances, the temperature of the evaporation vessel can be fixed (i.e., can be maintained) during the concentrating. In other instances, the temperature of the evaporation vessel can be varied during the concentrating (i.e., can be increased or can be decreased from the initial temperature).

In some instances, the rotating blade can rotate at an initial speed of about 15 rpm to about 105 rpm. In other instances, the initial speed can be about 16 rpm to about 104 rpm, about 17 rpm to about 103 rpm, about 18 rpm to about 102 rpm, about 19 rpm to about 101 rpm, about 20 rpm to about 100 rpm, about 21 rpm to about 99 rpm, about 22 rpm to about 98 rpm, about 23 rpm to about 97 rpm, about 24 rpm to about 96 rpm, about 25 rpm to about 95 rpm, about 26 rpm to about 94 rpm, about 27 rpm to about 93 rpm, about 28 rpm to about 92 rpm, about 29 rpm to about 91 rpm, about 30 rpm to about 90 rpm, about 31 rpm to about 89 rpm, about 32 rpm to about 88 rpm, about 33 rpm to about 87 rpm, about 34 rpm to about 86 rpm, about 35 rpm to about 85 rpm, about 36 rpm to about 84 rpm, about 37 rpm to about 83 rpm, about 38 rpm to about 82 rpm, about 39 rpm to about 81 rpm, about 40 rpm to about 80 rpm, about 41 rpm to about 79 rpm, about 42 rpm to about 78 rpm, about 43 rpm to about 77 rpm, about 44 rpm to about 76 rpm, about 45 rpm to about 75 rpm, about 46 rpm to about 74 rpm, about 47 rpm to about 73 rpm, about 48 rpm to about 72 rpm, about 49 rpm to about 71 rpm, about 50 rpm to about 70 rpm, about 51 rpm to about 69 rpm, about 52 rpm to about 68 rpm, about 53 rpm to about 67 rpm, about 54 rpm to about 66 rpm, about 55 rpm to about 65 rpm, about 56 rpm to about 64 rpm, about 57 rpm to about 63 rpm, about 58 rpm to about 62 rpm, about 59 rpm to about 61 rpm, or about 60 rpm. In yet other instances, the initial speed can be about 15 rpm to about 20 rpm, about 20 rpm to about 25 rpm, about 25 rpm to about 30 rpm, about 30 rpm to about 35 rpm, about 35 rpm to about 40 rpm, about 40 rpm to about 45 rpm, about 45 rpm to about 50 rpm, about 50 rpm to about 55 rpm, about 55 rpm to about 60 rpm, about 60 rpm to about 65 rpm, about 65 rpm to about 70 rpm, about 70 rpm to about 75 rpm, about 75 rpm to about 80 rpm, about 80 rpm to about 85 rpm, about 85 rpm to about 90 rpm, about 90 rpm to about 95 rpm, about 95 rpm to about 100 rpm, or about 100 rpm to about 105 rpm. In yet other instances, the initial speed can be about 15 rpm, about 20 rpm, about 25 rpm, about 30 rpm, about 35 rpm, about 40 rpm, about 45 rpm, about 50 rpm, about 55 rpm, about 60 rpm, about 65 rpm, about 70 rpm, about 75 rpm, about 80 rpm, about 85 rpm, about 90 rpm, about 95 rpm, about 100 rpm or about 105 rpm.

In some instances, the rotating blade speed can be fixed (i.e., can be maintained) during the concentrating. In other instances, the rotating blade speed can be varied during the concentrating (i.e., can be increased or can be decreased from the initial speed).

The method also can include a step (b) of passing a gas, such as an inert gas, through the evaporation vessel. In some instances, the gas can be nitrogen. In some instances, the gas can be at an initial sweep pressure of about 1.5 psi to about 5.0 psi. In other instances, the initial sweep pressure can be about 1.6 psi to about 4.9 psi, about 1.7 psi to about 4.8 psi, about 1.8 psi to about 4.7 psi, about 1.9 psi to about 4.6 psi, about 2.0 psi to about 4.5 psi, about 2.1 psi to about 4.4 psi, about 2.2 psi to about 4.3 psi, about 2.3 psi to about 4.2 psi, about 2.4 psi to about 4.1 psi, about 2.5 psi to about 4.0 psi, about 2.6 psi to about 3.9 psi, about 2.7 psi to about 3.8 psi, about 2.8 psi to about 3.7 psi, about 2.9 psi to about 3.6 psi, about 3.0 psi to about 3.5 psi, about 3.1 psi to about 3.2 psi, or about 3.3 psi to about 3.4 psi. In other instances, the initial sweep pressure can be about 1.5 psi to about 2.0 psi, about 2.0 psi to about 2.5 psi, about 2.5 psi to about 3.0 psi, about 3.0 psi to about 3.5 psi, about 3.5 psi to about 4.0 psi, about 4.0 psi to about 4.5 psi, or about 4.5 psi to about 5.0 psi. In yet other instances, the initial sweep pressure can be about 1.5 psi, about 1.6 psi, about 1.7 psi, about 1.8 psi, about 1.9 psi, about 2.0 psi, about 2.1 psi, about 2.2 psi, about 2.3 psi, about 2.4 psi, about 2.5 psi, about 2.6 psi, about 2.7, about 2.8 psi, about 2.9 psi, about 3.0 psi, about 3.1 psi, about 3.2 psi, about 3.3 psi, about 3.4 psi, about 3.5 psi, about 3.6 psi, about 3.7 psi, about 3.8 psi, about 3.9 psi, about 4.0 psi, about 4.1 psi, about 4.2 psi, about 4.3 psi, about 4.4 psi, about 4.5 psi, about 4.6 psi, about 4.7 psi, about 4.8 psi, about 4.9 psi or about 5.0 psi.

In some instances, the sweep pressure can be fixed (i.e., can be maintained) during the concentrating. In other instances, the sweep pressure can be varied during the concentrating (i.e., can be increased or can be decreased from the initial pressure).

The method also can include a step (c) of condensing and collecting a volatile liquid from the solution in a condenser in fluid communication with the evaporation vessel, where the condenser is at an initial temperature.

In some instances, the condenser is an internal condenser (i.e., within the evaporation vessel). In other instances, the condenser is an external condenser (i.e., separate from the evaporation vessel). In some instances, the initial temperature of the condenser is about 0° C.

In some instances, the temperature of the condenser can be fixed (i.e., can be maintained) during the concentrating. In other instances, the temperature of the condenser can be varied during the concentrating (i.e., can be increased or can be decreased from the initial temperature).

The method also can include a step (d) of repeating steps (a) to (c) at least one additional time, whereby the solution has a final oligonucleotide concentration of ≥about 200 mg/mL. In other instances, the final oligonucleotide concentration can be about 200 mg/mL to about 400 mg/mL. In yet other instances, the final oligonucleotide concentration can be about 210 mg/mL to about 390 mg/mL, about 220 mg/mL to about 380 mg/mL, about 230 mg/mL to about 370 mg/mL, about 240 mg/mL to about 360 mg/mL, about 250 mg/mL to about 350 gm/mL, about 260 mg/mL to about 340 mg/mL, about 270 mg/mL to about 330 mg/mL, about 280 mg/mL to about 320 mg/mL, about 290 mg/mL to about 310 mg/mL or about 300 mg. In yet other instances, the final oligonucleotide concentration can be about 200 mg/mL, about 210 mg/mL, about 220 mg/mL, about 230 mg/mL, about 240 mg/mL, about 250 mg/mL, about 260 mg/mL, about 270 mg/mL, about 280 mg/mL, about 290 mg/mL, about 300 mg/mL, about 310 mg/mL, about 320 mg/mL, about 330 mg/mL, about 340 mg/mL, about 350 mg/mL, about 360 mg/mL, about 370 mg/mL, about 380 mg/mL, about 390 mg/mL or about 400 mg/mL. In still other instances, the final oligonucleotide concentration is ≤about 450 mg/mL. In yet other instances in the methods above, the final oligonucleotide concentration can be ≤about 500 mg/mL.

In some instances, the final volume of the oligonucleotide-containing solution can be ≤about 2 mL.

### “Single-Pass” IE

Briefly, a method of concentrating an oligonucleotide-containing solution via IE can include a step (a) of loading an initial volume of the solution into an evaporation vessel, where the evaporation vessel is at an initial pressure and at an initial temperature.

In some instances, the initial oligonucleotide concentration in the oligonucleotide-containing solution can be ≤20 mg/mL. In other instances, the initial oligonucleotide concentration can be between about 5 mg/mL to about 20 mg/mL or about 10 mg/mL to about 15 mg/mL. In yet other instances, the initial oligonucleotide concentration can be about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/L, about 18 mg/mL, about 19 mg/mL or about 20 mg/mL. In yet other instances, the initial oligonucleotide concentration can be >about 20 mg/mL but <about 30 mg/mL. In some instances, the initial oligonucleotide concentration can be >about 30 mg/mL as long as it is a large enough volume and low enough viscosity to flow through the system (e.g., even >about 100 mg/mL in appropriate instances).

In some instances, the pH of the oligonucleotide-containing solution can be about 6.0, about 6.1, about 6.2, about 6.3, about 6.4, about 6.5, about 6.6, about 6.7, about 6.8, about 6.9 or about 7.0.

In some instances, the initial volume of oligonucleotide-containing solution is about 1 mL to about 10 mL. In other instances, the initial volume can be about 2 mL to about 9 mL, about 3 mL to about 8 mL, about 4 mL to about 7 mL, or about 5 mL to about 6 mL. In yet other instances, the initial volume can be about 1 mL, about 2 mL, about 3 mL, about 4 mL, about 5 mL, about 6 mL, about 7 mL, about 8 mL, about 9 mL or about 10 mL. In even other instances, and depending upon the size of the evaporation vessel, the initial volume of oligonucleotide-containing solution can be >10 mL.

In some instances, the initial pressure of the evaporation vessel can be about 20 torr to about 30 torr. In other instances, the initial pressure can be about 20 torr to about 22 torr, about 22 torr to about 24 torr, about 24 torr to about 26 torr, about 26 torr to about 28 torr, or about 28 torr to about 30 torr. In yet other instances, the initial pressure can be about 20 torr, about 21 torr, about 22 torr, about 23 torr, about 24 torr, about 25 torr, about 26 torr, about 27 torr, about 28 torr, about 29 torr or about 30 torr.

In some instances, the pressure of the evaporation vessel can be fixed (i.e., can be maintained) during the concentrating. In other instances, the pressure of the evaporation vessel can be varied during the concentrating (i.e., can be increased or can be decreased from the initial pressure).

In some instances, the initial temperature of the evaporation vessel can be about 55° C. to about 65° C. In other instances, the initial temperature can be about 56° C. to about 64° C., about 57° C. to about 63° C., about 58° C. to about 62° C., about 59° C. to about 61° C., or about 60° C. In yet other instances, the initial temperature can be about 55° C. to about 57° C., about 57° C. to about 59° C., about 59° C. to about 61° C., about 61° C. to about 63° C., or about 63° C. to about 65° C. In yet other instances, the initial temperature can be about 55° C., about 56° C., about 57° C., about 58° C., about 59° C., about 60° C., about 61° C., about 62° C., about 63° C., about 64° C. or about 65° C.

In some instances, the temperature of the evaporation vessel can be fixed (i.e., can be maintained) during the concentrating. In other instances, the temperature of the evaporation vessel can be varied during the concentrating (i.e., can be increased or can be decreased from the initial temperature).

The method also can include a step (b) of boiling the solution in the evaporation vessel for a period of time.

In some instances, the initial period of time for boiling can be for about 4 min to about 4.5 min. In other instances, the initial period of time can be about 4.1 min to about 4.4 min or about 4.2 min to about 4.3 min. In yet other instances, the initial period of time can be about 4 min to 4.1 min, about 4.1 min to about 4.2 min, about 4.2 min to about 4.3 min, about 4.3 min to about 4.4 min, or about 4.4 min to about 4.5 min. In yet other instances, the initial period of time can be about 4 min, about 4.1 min, about 4.2 min, about 4.3 min, about 4.4 min or about 4.5 min, especially about 4.25 min.

The method also can include a step (c) of drawing water vapor from the evaporation vessel via a condenser, where the condenser is an initial temperature, whereby the solution has a final oligonucleotide concentration of ≥about 200 mg/mL.

In some instances, the condenser is an internal condenser (i.e., within the evaporation vessel). In other instances, the condenser is an external condenser (i.e., separate from the evaporation vessel). In some instances, the initial temperature of the condenser is about 0° C.

In some instances, the initial temperature of the condenser can be fixed (i.e., can be maintained) during the concentrating. In other instances, the initial temperature of the condenser can be varied during the concentrating (i.e., can be increased or can be decreased from the initial temperature).

In some instances, the method also can include a step (d) of transferring the solution having a final oligonucleotide concentration of ≥about 200 mg/mL from the evaporation vessel to, for example, a collection vessel.

In some instances, the method also can include a step of (e) repeating steps (a) to (d) on a fresh initial volume of the oligonucleotide-containing solution.

In some instances, the oligonucleotide is a ss oligonucleotide. In other instances, the oligonucleotide is a ds oligonucleotide.

In some instances, the final oligonucleotide concentration in the oligonucleotide-containing solution can be ≥about 200 mg/mL. In other instances, the final oligonucleotide concentration can be about 200 mg/mL to about 400 mg/mL. In yet other instances, the final oligonucleotide concentration can be about 210 mg/mL to about 390 mg/mL, about 220 mg/ml to about 380 mg/mL, about 230 mg/mL to about 370 mg/mL, about 240 mg/mL to about 360 mg/mL, about 250 mg/mL to about 350 gm/mL, about 260 mg/mL to about 340 mg/mL, about 270 mg/mL to about 330 mg/mL, about 280 mg/mL to about 320 mg/mL, about 290 mg/mL to about 310 mg/mL or about 300 mg. In yet other instances, the final oligonucleotide concentration can be about 200 mg/mL, about 210 mg/mL, about 220 mg/mL, about 230 mg/mL, about 240 mg/mL, about 250 mg/mL, about 260 mg/mL, about 270 mg/mL, about 280 mg/mL, about 290 mg/mL, about 300 mg/mL, about 310 mg/mL, about 320 mg/mL, about 330 mg/mL, about 340 mg/mL, about 350 mg/mL, about 360 mg/mL, about 370 mg/mL, about 380 mg/mL, about 390 mg/mL or about 400 mg/mL. In still other instances, the final oligonucleotide concentration can be ≤about 450 mg/mL. In yet other instances in the methods above, the final oligonucleotide concentration can be ≤about 500 mg/mL.

In some instances, the final volume of the oligonucleotide-containing solution can be ≤about 2 mL.

### EXAMPLES

The following non-limiting examples are offered for purposes of illustration, not limitation.

### Example 1: Concentrating a Ds Oligonucleotide-Containing Solution Via a Combination of TFF and WFE

Purpose: To assess the effect of a combination of TFF and WFE on concentrating a ds oligonucleotide-containing solution.

### Methods

Oligonucleotide-Containing Solution: The ds oligonucleotide used was a sodium salt duplex consisting of a 36-nucleotide sense strand containing GalNAc sugars on positions 28-30 (SEQ ID NO:3) complexed to a 22-nucleotide antisense strand (SEQ ID NO:4). The concentration of the ds oligonucleotide in H₂O was 20 mg/mL (determined by a UV assay), with fresh aliquots taken as starting material.

TFF: TFF was performed on the 20 mg/mL ds oligonucleotide-containing solution using a Pendotech® TFF System with a 2 L retentate reservoir, 6.4 mm inner diameter tubing size (Masterflex® 96410-17), retentate and permeate balances (Ohaus) and a Quattroflow® 150 circulation pump (PSG). The membranes used are listed below in Table 2. The system and membranes were flushed and equilibrated with H₂O prior to use and then loaded with 765 mL of the 20 mg/mL ds oligonucleotide-containing solution in the retentate reservoir, for a loading of about 15.3 g of ds oligonucleotide. The concentrating experiment was run with a feed flow rate of 1 mL/min and a transmembrane pressure (TMP) of 30 psi. The run was stopped when the minimum volume of the retentate vessel was reached. The primary retentate was collected, followed by washes with about 60 mL and 100 mL of H₂O. The system was sanitized by flushing with 0.5 N NaOH for 1 hr and stored in 0.1 N NaOH.

TABLE 1
Filters Used for TFF of Oligonucleotide-Containing Solutions

| | | | | Surface | |
|---|---|---|---|---|---|
| Filter | Manu- | | | Area | MWCO |
| Name | facturer | Material | Lot # | (m²) | (kDa) |
| Centramate | Pall | Omega PES | 31279015R | 0.1 | 3 |
| T-Series | | | | | |
| Centramate | Pall | Omega PES | 31279016R | 0.1 | 3 |
| T-Series | | | | | |
| Centramate | Pall | Omega PES | 31279013R | 0.1 | 3 |
| T-Series | | | | | |

WFE: WFE was performed on a 2-inch diameter wiped-film (i.e., short path) molecular still (Pope Scientific Inc.), with modifications as shown in FIG. 1. The feed funnel was replaced with a 1 L syringe pump (Isco) to allow continuous feed flow, with a metering valve installed on the flow path of the feed to provide a positive back pressure in the syringe pump. The residual collection flask was replaced by perfluoroalkoxy (PFA) tubing (½-inch outer diameter, ⅜-inch inner diameter) connected by a Swagelok® manual valve to a size #35 Ace-thread glass bottle. A long piece of 1/16-inch tubing was inserted in the ½-inch PFA tubing, extending from above the manual valve up to the wiped film still body to help the liquid flow through a narrow opening on the top part of the tubing, where it connected to a glass connector. A nitrogen line was connected as indicated in FIG. 1 to alleviate condensation forming near the collection tubing. The nitrogen line was regulated at 2 psi and was connected to a metering valve to adjust the nitrogen flow. The system was connected to a vacuum pump through a vacuum controller. A bath of hot glycol/water was recirculated through the system to provide heat for evaporation, and another bath of cold glycol/water for condensing the water vapor. There were two configurations for connecting the heating/cooling liquid to the system: (1) hot liquid flowing through the outer jacket of the still, and cold liquid first flowing through the center condensing rod and then flowing through the external condenser, or (2) hot liquid first flowing through the outer jacket of the still and then the center rod, and cold liquid only flowing through the external condenser. Configuration 1 is short-path evaporation, suitable for high throughput and low vacuum, while configuration 2 is more suitable for having better control of extent of evaporation.

For the concentrating experiments, the ds oligonucleotide-containing solution was loaded into the syringe pump and fed into the apparatus at a set flow rate. The temperature of the heating jacket was set to 55° C., the temperature of the external condenser to 0° C., the rotation of the wiper was set to 20 rpm, and the 2 psi nitrogen valve was open to a mark of 5. The nitrogen sweep was adjusted so that there was no water condensation on the arm connected to the residual flask and at the same time still allows the pressure can be maintained at target set point. The vacuum pressure and feed flow rates varied slightly between experiments, with vacuum pressure values in the range of 20-32 torr and feed flow rates in the range of 3.4-3.6 mL/min. Values were varied as needed to prevent or alleviate any observed gel formation.

Density and Concentration Assays: Concentrated samples were filtered with 0.22 μm filters (Millipore; Burlington, MA, Steriflip 50 mL with 0.22 μm Durapore PVDF membranes) prior to density and concentration measurements.

Density measurements were recorded using a DMA 4100 M Density Meter (Anton Paar; Ashland, VA) at 20° C. Density measurements were used to make gravimetric dilutions of oligonucleotide-containing solutions for concentration measurements.

Concentration values were generated using a UV assay. Samples were diluted gravimetrically to approximately 2 mg/mL then further to 0.02 mg/mL for measurement using a Cary UV-Vis Multicell Peltier instrument. Measurements were taken of each sample in triplicate using 1 cm pathlength cuvettes, with H₂O as reference. The following equation was used to generate an average concentration value, where Abs was the average absorbance value at 258 nm from triplicate measurements, V was the volume of the of the first dilution, density was the measured density of the concentrated solution, MW was the molecular weight of the free acid form of the molecule (20675 g/mol), DF was the dilution factor of the second dilution, W was the sample weight of the concentrated sample, MEC was the molar extinction coefficient (548000 M⁻¹cm⁻¹), Path Length was 1 cm, and Purity was determined from non-denaturing UPLC.

$$Assay ( mg / ml ) = \frac{Abs \times V \times Density \times MW \times DF}{W \times MEC \times Path Length} \times \frac{100 mg / g}{1000 mL / L} \times Purity$$

Denaturing and Non-Denaturing UPLC Purity Assays: Denaturing and non-denaturing UPLC assays were used to analyze and compare oligonucleotide purity before and after concentration experiments. Samples were diluted to about 2 mg/mL for measurement.

The denaturing assay was performed with a Waters Acquity UPLC Peptide BEH C18 column (1.7 μm, 2.1×100 mm, part number 186003686), a column temperature of 80° C., and a detection of 259 nm at 20 Hz. Mobile phase A was 28 mM DIPEA with 100 mM HFIP, and mobile phase B was a 90:10 mixture of methanol: IPA, with a method gradient listed in Table 2.

TABLE 2
Gradient for Denaturing UPLC.

| | Time | Flow | | | |
|---|---|---|---|---|---|
| | (min) | (mL/min) | % A | % B | Curve |
| 1 | Initial | 0.400 | 85.0 | 15.0 | Initial |
| 2 | 1.00 | 0.400 | 85.0 | 15.0 | 6 |
| 3 | 15.00 | 0.400 | 79.0 | 21.0 | 6 |
| 4 | 15.10 | 0.400 | 40.0 | 60.0 | 6 |
| 5 | 17.00 | 0.400 | 40.0 | 60.0 | 6 |
| 6 | 17.10 | 0.400 | 85.0 | 15.0 | 6 |
| 7 | 20.00 | 0.400 | 85.0 | 15.0 | 6 |

The non-denaturing assay was performed with a Waters XBridge BEH C18 column (2.5 μm, 2.1×50 mm, part number 186006029), a column temperature of 20° C., and a detection of 260 nm at 20 Hz. Mobile phase A was 95 mM TEA with 14 mM HFIP, and mobile phase B was a 75:25 mixture of methanol: IPA, with a method gradient listed in Table 4.

TABLE 3
Gradient for Non-Denaturing UPLC.

| | Time | Flow | | | |
|---|---|---|---|---|---|
| | (min) | (mL/min) | % A | % B | Curve |
| 1 | Initial | 0.250 | 95.0 | 5.0 | Initial |
| 2 | 3.00 | 0.250 | 95.0 | 5.0 | 6 |
| 3 | 12.00 | 0.250 | 67.0 | 33.0 | 6 |
| 4 | 13.00 | 0.250 | 40.0 | 60.0 | 6 |
| 5 | 15.00 | 0.250 | 40.0 | 60.0 | 6 |
| 6 | 15.10 | 0.250 | 95.0 | 5.0 | 6 |
| 7 | 19.00 | 0.250 | 95.0 | 5.0 | 6 |

Viscosity Assay: A viscosity assay was performed on samples collected at different concentrations from additional WFE experiments. For samples less than 350 mg/mL, viscosity measurements were taken at 20° C. using an Anton Paar Lovis2000M Viscometer. A 1.8 mm capillary and 1.5 mm steel balls were used for measurement. Viscosities of samples greater than 350 mg/mL were measured at 20° C. using a RheoSense VROC® Initium Viscometer.

Results: ds oligonucleotide-containing solution concentrations following various treatments are shown below in Table 4.

TABLE 4
Final ds Oligonucleotide Concentration of 20 mg/mL Oligonucleotide-
Containing Solution Following Treatment.

| | | Final ds Oligonucleotide |
|---|---|---|
| Sample | Treatment | Concentration (mg/mL) |
| 1 | none | 20 |
| 2 | TFF only | 64 |
| 3 | TFF then WFE | 280 |

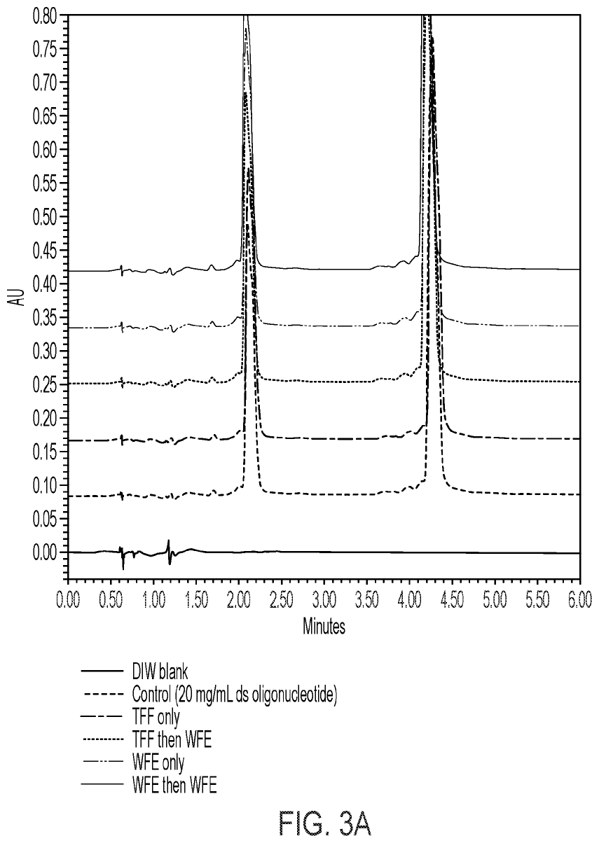

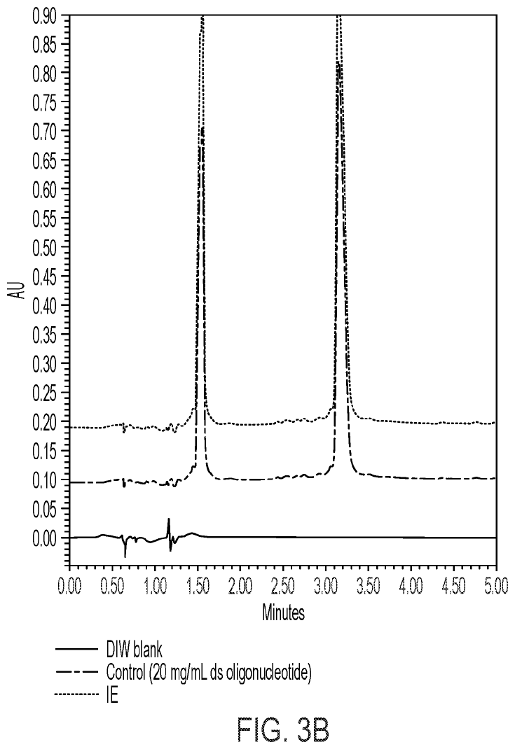

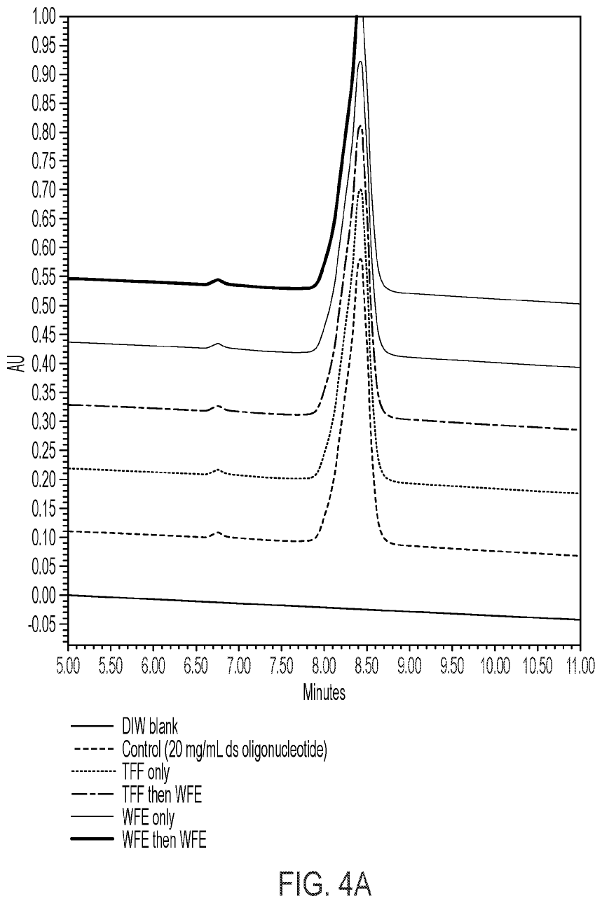

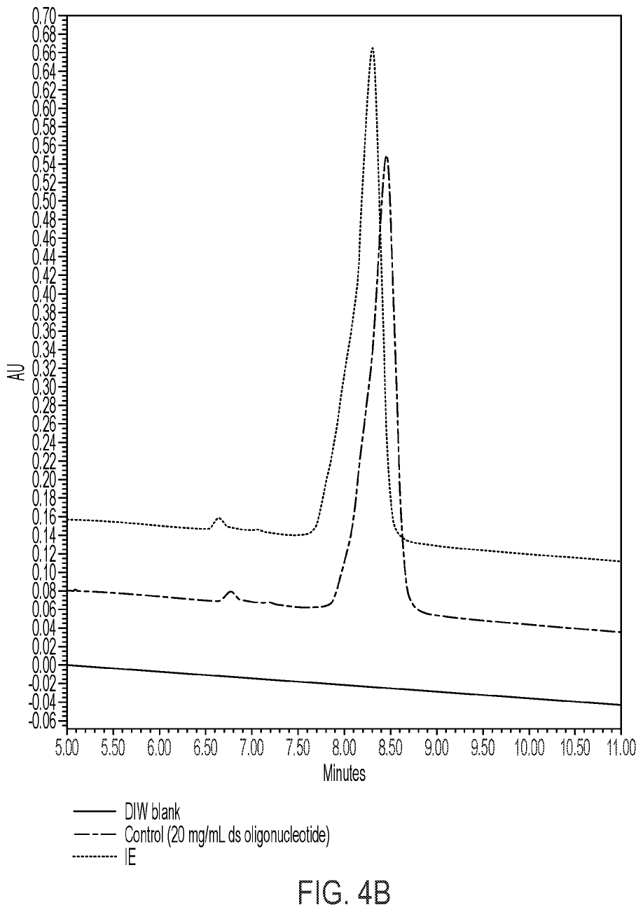

The denaturing assay results show two main peaks were observed correlating to both the sense and antisense single strands of the ds oligonucleotide (FIG. 3A). No significant difference in impurities was observed between the initial sample and the concentrated samples. Additionally, the non-denaturing assay results show a main peak correlating to the duplex, and all possessed a similar level of purity (~98.5-98.7 area %, with ~0.8-1 area % residual antisense strand) (FIG. 4A). These results suggest that there was no significant change in chemical purity due to the concentrating methods.

Here, a two-pass method was needed to achieve ds oligonucleotide concentrations of about 300 mg/mL.

### Example 2: Concentrating a Ds Oligonucleotide-Containing Solution Via WFE

Purpose: To assess the effect of WFE alone and repeated WFE (“double pass” WFE) on concentrating a ds oligonucleotide-containing solution.

### Methods

Oligonucleotide-Containing Solution: the oligonucleotide-containing solution was as described above in Example 1.

WFE: WFE was performed as described above in Example 1; however, in some instances WFE was performed twice on a sample.

Density and Concentration Assays: Density and concentration assays were performed as described above in Example 1.

Denaturing and Non-Denaturing UPLC Purity Assays: UPLC purity assays were performed as described above in Example 1.

Viscosity Assay: A viscosity assay was performed as described above in Example 1.

Results: ds oligonucleotide-containing solution concentrations following various treatments are shown below in Table 5.

TABLE 5
Final ds Oligonucleotide Concentration of 20 mg/mL Oligonucleotide-
Containing Solution Following Treatment.

| | | Final ds Oligonucleotide |
|---|---|---|
| Sample | Treatment | Concentration (mg/mL) |
| 1 | none | 20 |
| 4 | WFE only | 115 |
| 5 | WFE then WFE | 346 |

When starting from an intermediate ds oligonucleotide concentration in the range of about 80 mg/mL to about 115 mg/mL (achieved by a single pass of WFE), final ds oligonucleotide concentrations >300 mg/mL were reliably achieved by “dual pass” WFE.

The denaturing assay results show two main peaks were observed correlating to both the sense and antisense single strands of the ds oligonucleotide (FIG. 3A). No significant difference in impurities was observed between the initial sample and the concentrated samples. Additionally, the non-denaturing assay results show a main peak correlating to the duplex, and all possessed a similar level of purity (~98.5-98.7 area %, with ~0.8-1 area % residual antisense strand) (FIG. 4A). These results suggest that there was no significant change in chemical purity due to the concentrating methods.

### Example 3: Concentrating a Ds Oligonucleotide-Containing Solution Via IE

Purpose: To assess the effect of IE alone on concentrating a ds oligonucleotide-containing solution.

### Methods

Oligonucleotide-Containing Solution: the oligonucleotide-containing solution is as described above in Example 1.

IE: IE was performed on an automated, custom-built setup as shown in FIG. 2. For the concentrating experiments, the oligonucleotide-containing solution was loaded into the syringe pump (1 L, Isco), and 5 mL at a time was charged into the evaporation vessel. The temperature of the hot heat transfer solution (glycol/water) was set to 60° C., and the vacuum was set to 25 torr, while water vapor was collected on an external condenser. After a specified boiling time of 4.25 min, the concentrated solution was transferred to a collection container via a pressure difference generated by temporarily releasing the vacuum in the evaporator for 12 sec.

Density and Concentration Assays: Density and concentration assays were performed as described above in Example 1.

Denaturing and Non-Denaturing UPLC Purity Assays: UPLC purity assays were performed as described above in Example 1.

Viscosity Assay: A viscosity assay was performed as described above in Example 1.

Results: ds oligonucleotide-containing solution concentrations following treatment is shown below in Table 6.

TABLE 6
Final ds Oligonucleotide Concentration of 20 mg/mL Oligonucleotide-
Containing Solution Following Treatment.

| | | Final ds Oligonucleotide |
|---|---|---|
| Sample | Treatment | Concentration (mg/mL) |
| 1 | none | 20 |
| 6 | IE only | 359* |

*Due to low sample volume, an accurate density measurement could not be taken. The concentration value shown is calculated using the density from Sample 5. Using a range of densities generated from the same run, the actual concentration is in the range of 353-365 mg/mL.

The denaturing assay results show two main peaks were observed correlating to both the sense and antisense single strands of the ds oligonucleotide (FIG. 3B). No significant difference in impurities was observed between the initial sample and the concentrated samples. Additionally, the non-denaturing assay results show a main peak correlating to the duplex, and all possessed a similar level of purity (~98.5-98.7 area %, with ~0.8-1 area % residual antisense strand) (FIG. 4B). These results suggest that there was no significant change in chemical purity due to the concentrating methods.

### SEQUENCE LISTING

The following nucleotide and/or amino acid sequences are referred to in the disclosure above and are provided below for reference.

Synthetic oligonucleotide 1 (36 nt)
SEQ ID NO: 1
UCAAAAUGGAAGGUUAUACAGCAGCCGAAAGGCUGC
Synthetic oligonucleotide 2 (22 nt)
SEQ ID NO: 2
UGUAUAACCUUCCAUUUUGAGG
Synthetic oligonucleotide 3 (36 nt)
SEQ ID NO: 3
[mUs][mC][mA][mA][mA][mA][mU][fG][fG][fA][fA]
[mG][mG][mU][mU][mA][mU][mA][mC][mA][mG][mC]
[mA][mG][mC][mC][mG][ademA-GalNAc][ademA-
GalNAc][ademA-GalNAc][mG][mG][mC][mU][mG][mC]
Synthetic oligonucleotide 4 (22 nt)
SEQ ID NO: 4
[MePhosphonate-4O-mUs][fGs][fUs][fA][fU][mA]
[fA][mC][mC][fU][mU][mC][mC][fA][mU][mU][mU]
[mU][mG][mAs][mGs][mG]

1. A method of concentrating oligonucleotides in solution, the method comprising the steps of:
(a). flowing an oligonucleotide-containing solution through an evaporation vessel, wherein the solution has an initial oligonucleotide concentration of ≤about 20 mg/mL, wherein the evaporation vessel comprises a rotatable blade, a means for heating and a means for pressurizing, wherein the rotatable blade wipes the solution into a thin film around an internal surface of the evaporation vessel, and wherein the evaporation vessel is at an initial pressure and at an initial temperature;
(b). passing a gas through the evaporation vessel;
(c). condensing and collecting a volatile liquid from the solution in a condenser in fluid communication with the evaporation vessel, wherein the condenser is at an initial temperature; and
(d). repeating steps (a) to (c) at least one additional time, whereby the solution has a final oligonucleotide concentration of ≥about 200 mg/mL.

2. The method of claim 1, wherein the oligonucleotide-containing solution is at a pH between about 6 to about 7.

3. The method of claim 1, wherein the oligonucleotide is single stranded (ss).

4. The method of claim 1, wherein the oligonucleotide is double stranded (ds).

5. The method of claim 1, wherein the oligonucleotide-containing solution is flowed through the evaporation vessel at an initial flow rate of about 0.5 mL/min to about 4.5 mL/min.

6. The method of claim 5, wherein the initial flow rate is about 3.4 mL/min to about 3.6 mL/min.

7. The method of claim 1, wherein the rotatable blade rotates at an initial speed of about 15 rpm to about 105 rpm.

8. The method of claim 7, wherein the initial speed is about 20 rpm.

9. The method of claim 1, wherein the initial pressure of the evaporation vessel is about 10 torr to about 65 torr.

10. The method of claim 9, wherein the initial pressure is about 20 torr to about 32 torr.

11. The method of claim 1, wherein the initial temperature of the evaporation vessel is about 35° C. to about 60° C.

12. The method of claim 11, wherein the initial temperature is about 55° C.

13. The method of claim 1, wherein the gas is at an initial sweep pressure of about 1.5 psi to about 5 psi.

14. The method of claim 13, wherein the initial sweep pressure is about 2 psi.

15. The method of claim 13, wherein the gas is nitrogen gas.

16. The method of claim 1, wherein the initial temperature of the condenser is about 0° C.

17. The method of claim 1, wherein steps (a) to (c) are repeated once.

18. The method of claim 1, wherein the final oligonucleotide concentration is about 200 mg/mL to about 400 mg/mL.

19. The method of claim 1, wherein the final oligonucleotide concentration is ≤about 450 mg/mL.

20. The method of claim 1, wherein the final oligonucleotide concentration is ≤about 500 mg/mL.

21-39. (canceled)

40. An apparatus for concentrating biomolecules in a solution, the apparatus comprising:
(i). an evaporation vessel, wherein the evaporation vessel comprises a means for heating the evaporation vessel and a means for pressurizing the evaporation vessel;
(ii). a first reservoir in fluid communication with the evaporation vessel, wherein the first reservoir is a feed for an unconcentrated biomolecule-containing solution;
(iii). a second reservoir in fluid communication with the evaporation vessel, wherein the second reservoir is a collector for a concentrated biomolecule-containing solution; and
(iv). a condenser in fluid communication with the evaporation vessel, wherein the condenser comprising a means for cooling the condenser and a means for pressurizing the condenser.
